(19) United States
(12) Patent Application Publication
(10) Pub. No.: US 2026/0028300 A1
(43) Pub. Date: Jan. 29, 2026

(54) A PROCESS OF PREPARING DIVANILLYLMETHANE

(71) Applicant: ADITYA BIRLA CHEMICALS (THAILAND) LTD. (ADVANCED MATERIALS), BANGKOK (TH)

(72) Inventors: PRADIP KUMAR DUBEY, RAYONG (TH); BHARAT SINGH, MAHARASHTRA (IN); THIPA NAIYAWAT, RAYONG (TH); KAMONICHA CHAYTHAWEEP, RAYONG (TH); DAPAWAN KUNWONG, RAYONG (TH); WORAKARN CHAMONGKOLPRADIT, RAYONG (TH)

(21) Appl. No.: 19/114,273

(22) PCT Filed: Sep. 21, 2023
(86) PCT No.: PCT/IB2023/059337

§ 371 Date: Mar. 21, 2025

(30) Foreign Application Priority Data
Sep. 22, 2022 (IN) 202211054170

(51) Int. Cl. C07C 45/75; C07C 45/79; C07C 45/80; C07C 45/86

(52) U.S. Cl. CPC C07C 45/75; C07C 45/79; C07C 45/80; C07C 45/86

(57) ABSTRACT

The present disclosure provides a process for the synthesis of divanillylmethane. The process includes reacting vanilline with an aqueous solution of formaldehyde in the presence of an acid catalyst.

### FIELD OF THE INVENTION

The present disclosure relates to a process for the synthesis of divanillylmethane, a monomer for the preparation of an epoxy resin.

### BACKGROUND

Epoxy resins are known as very versatile thermosetting resins. Due to their unique characteristics including excellent chemical resistance, high moisture and solvent resistances, good thermal and dimensional stabilities, high adhesion strength, superior electrical properties, epoxy resins are used in a wide variety of applications such as adhesives, in high performance composites, coatings and electronics.

However, a major drawback with epoxy resins is that the components that are used for the manufacture of epoxy resins are obtained from petroleum products. It is estimated that almost 90% of the world's production of epoxy resin is based on the reaction of petroleum-based bisphenol A and epichlorohydrin, producing diglycidyl ether of bisphenol A.

The huge usage of fossil fuels is considered as a large contributor to the global warming and the climate change, and the fossil fuels are depleted. which in collection have encouraged scientists and researchers to find an alternative resources to produce polymeric materials. In addition, increasing health and the environmental concerns over using bisphenol A (BPA), due to its toxicity and carcinogenic effects, have been raised in recent decades. Therefore, there is an arousing interest in seeking green alternative to BPA for developing bio-based and eco-friendly epoxy resins.

Vanillin, a bio-based monomer derived from lignin, along with its derivatives has shown great potential for the production of high-performance epoxy resins due to its unique structure and good properties. Divanillylmethane is a good candidate that can replace Bisphenol A for the production of epoxy resins. However, there is a need for preparing divanillylmethane by a simple method with high yield.

### SUMMARY

A process for the synthesis of divanillylmethane is provided. The process comprises reacting vanilline with an aqueous solution of formaldehyde in the presence of an acid catalyst.

### DETAILED DESCRIPTION

For the purpose of promoting an understanding of the principles of the disclosure, reference will now be made to embodiments and specific language will be used to describe the same. It will nevertheless be understood that no limitation of the scope of the disclosure is thereby intended, such alterations and further modifications in the disclosed composition and method, and such further applications of the principles of the disclosure therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “one embodiment”, “an embodiment” or similar language means that a particular feature, structure, or characteristic described in connection with the embodiment is included in at least one embodiment of the present disclosure. Thus, appearances of the phrase “in one embodiment”, “in an embodiment” and similar language throughout this specification may, but do not necessarily, all refer to the same embodiment.

The terms “a,” “an,”, and “the” are used to refer to “one or more” (i.e. to at least one) of the grammatical object of the article.

The present disclosure relates to a process of preparing divanillylmethane or 4,4-dihydroxy-5,5-dimethoxy-3,3-methanediyldibenzaldehyde of Formula I.

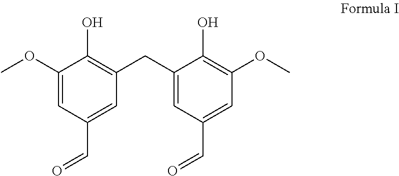

The process comprises reacting vanillin with formaldehyde in presence of an acid catalyst.

In accordance with an aspect, any strong acid catalyst may be used. The acid catalyst includes but is not limited to phosphoric acid, or sulphuric acid. The acid catalyst is phosphoric acid having a concentration in the range from 70% to 80%. In accordance with an aspect, the acid catalyst is phosphoric acid having a concentration in the range of 75% to 80%.

The process comprises preparing a mixture of vanillin and the acid catalyst and heating the mixture of vanillin and acid catalyst to a temperature in the range of 85-100° C. To said mixture of vanillin and acid catalyst an aqueous solution of formaldehyde is added to obtain a reaction mixture. The reaction mixture is heated to a temperature in the range of 110 to 115° C. In accordance with an aspect the aqueous solution of formaldehyde is added in a dropwise manner. The reaction mixture is allowed to react at a temperature in a range of 110 to 115° C. for 7 to 10 hours. In accordance with an aspect the reaction mixture is allowed to react for 8 hours to obtain a crude mixture comprising divanillylmethane, unreacted vanillin and the acid catalyst.

In accordance with an aspect, vanillin has purity in the range of 95 to 99.99%. In accordance with an embodiment vanillin has a purity of 99%.

In accordance with an aspect, the aqueous formaldehyde solution that is reacted with vanillin has a concentration in the range of 30 to 40% wt/wt. Preferably the formaldehyde solution has a concentration in the range of 35% to 40% wt/wt.

In accordance with an aspect, the molar ratio of vanillin to formaldehyde is in the range of 2-6. In accordance with an aspect, the molar ratio of phosphoric acid to formaldehyde is in a range of 3-5. In an alternate embodiment, the molar ratio of sulfuric acid to formaldehyde is in a range of 0.2-0.5.

The process further comprises of recovering divanillylmethane from the crude mixture. Divanillylmethane, is recovered from the crude mixture, by a process comprising the steps:
separating acid catalyst,
neutralizing the trace amount of acid catalyst with a base,
dissolving unreacted vanillin by a solvent to obtain a vanillin soluble liquid,
separating the solid from the vanillin soluble liquid to obtain a first solid extract,
removing traces of vanillin from the first solid extract by water extraction to obtain a second solid extract, and
removing the solvent and water from the second solid extract to obtain divanillylmethane.

In the first step of the process in recovering divanillylmethane, acid catalyst is recovered from the crude mixture. For recovering the acid catalyst, the crude mixture is allowed to cool to a temperature in a range of 70 to 80° C. Upon cooling acid catalyst settles down in the second/aqueous phase below the organic phase. The acid catalyst can be physically separated from the organic phase.

In the next step, traces of the acid catalyst remaining in the organic phase are neutralized with a base. In accordance with an aspect, sodium carbonate solution is added to get a pH in a range of 6 to 6.5, to neutralize the traces of the acid catalyst.

In the next step of the process divanillylmethane is separated from the unreacted vanillin using a solvent extraction method. The solvent extraction method comprises of dissolving unreacted vanillin by organic solvent and separating the solid from the vanillin soluble liquid to obtain a first solid extract. Any known solvent capable of dissolving vanillin may be used. In accordance with an aspect the solvents that may be used for solvent extraction method including but are not limited to acetone, and methyl isobutyl ketone. In accordance with an embodiment, the solvent is methyl isobutyl ketone. Any known method for separating the solid from the vanillin soluble liquid maybe used. In accordance with an aspect, filtration is used for separating the solid from the vanillin soluble liquid to obtain a first solid extract.

In the next step, traces of unreacted vanillin are removed from the first solid extract using water extraction. The process comprises of dissolving the first solid extract in water to dissolve the traces of vanillin and separating the solid to obtain a second solid extract. Any known method for separating the solid from the vanillin solution in water may be used. In accordance with an aspect, filtration is used for separating the solid from the vanillin solution in water.

In accordance with an aspect, solvent and water is removed from the second solid extract by distillation under vacuum to obtain divanillylmethane. The product obtained is light brown in color solid form and contains 80-97% divanillymethane and 3-20% vanillin.

The present disclosure also relates to a process for preparing an epoxy resin. The process comprises of reacting divanillylmethane of formula I with epichlorohydrin in the presence of a phase transfer catalyst such as benzyl triethylammonium chloride to obtain divanillylmethane epoxy resin having a formula II.

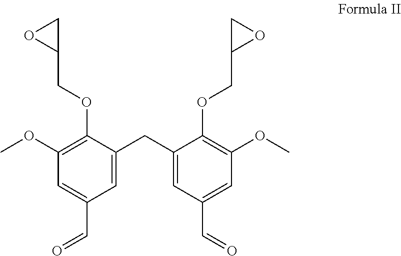

In accordance with an aspect, sodium hydroxide solution may be used in ring closing. In accordance with an aspect, divanillylmethane epoxy resin is a brown semi-solid having a monomer content of 75-80% and Epoxy Equivalent Weight of 233.2 g/eq.

The divanillylmethane epoxy resin may be cured using any known curing agent such as an amine curing agent, polyetheramine and latent curing agent.

In order that this disclosure may be better understood, the following examples are set forth. These examples are for the purpose of illustration only and the exact compositions, methods of preparation and embodiments shown are not limiting of the disclosure.

### Example 1

This example demonstrated the procedure for the condensation of vanillin and aqueous formaldehyde to obtain divanillylmethane (DVM). 700.0 g (4.60 mole) of vanillin and 292.9 g (2.30 mole) of 77% Phosphoric Acid (H₃PO₄) were placed in a round-bottom flask. Mixture was stirred and heated up to 80-85° C. 62.2 g (0.77 mole) of 37% formaldehyde solution was added dropwise into the mixture. Then the temperature was raised to 100° C. and kept for 8 hours. After completing reaction, it was cooled down to 80-85° C. Acid layer was separated. Crude was then neutralized by sodium bicarbonate solution. The obtained crude was a dark brown solid. Acetone was added equally to weight of crude and mixed at 30-40° C. for 90 min. Filtration was continued. Trace of acetone in solid part was evaporated at 85° C. under vacuum to obtain Extraction 1. The product obtained was a light brown solid. The product was analyzed by 1H-NMR, HPLC, GPC and DSC techniques. Yield of DVM in extracted solid was 34.5%. 1H-NMR confirmed DVM was achieved. The purity of DVM was 86.8% on GPC.

### Example 2-4

The same process as that used in Example 1 was used, except for the molar ratio of vanillin to formaldehyde. In particular, less molar ratio of vanillin to formaldehyde was used in exp 2-4. If purity of DVM was less than 85%, water extraction was continued. Water was added 90 part and mixed at 80-90° C. for 90 min (Extraction 2). The product was analyzed by 1H-NMR, HPLC, GPC and DSC techniques.

Table 1 provides the molar ratio of vanillin to formaldehyde used in examples 1 to 4 and yield of the product

TABLE 1

| | | | | Product after | | | |
|---|---|---|---|---|---|---|---|
| | | | | extraction 1 | | Product after | |
| | | | | | Yield | extraction 2 | |
| | | Crude after | | Purity | in | Purity | |
| | Mole | reaction | | of | solid | of | |
| | ratio | DVM | Vanillin | DVM | part | DVM | Yield |
| Experiment | V/F | (%) | (%) | (%) | (%) | (%) | (%) |
| ccc1 | 6 | 23.8 | 70.3 | 86.8 | 34.5 | — | — |
| 2 | 4 | 28.5 | 59.3 | 68.1 | 33.4 | 86.1 | 33.1 |
| 3 | 3 | 44.8 | 49.4 | 80.6 | 38.0 | 90.2 | 37.0 |
| 4 | 2 | 56.7 | 35.7 | 76.1 | 45.1 | 73.8 | — |

After reducing molar ratio of V/F, less vanillin remained in crude after reaction and yield after acetone extraction slightly increased. But purity of DVM after extraction decreased to 68-81% due to more vanillin remained. After continuing water extraction, vanillin was washed out and DVM purity could be enriched to 73-90%. Molar ratio of V/F at 3-4 could give DVM purity greater than 85% at yield 33-38%.

### Experiment 5-6

Different concentrations of phosphoric acid were used in experiment 3 condition. The results were shown in table 2.

TABLE 2

| | | | | Product after | |
|---|---|---|---|---|---|
| | H₃PO₄ | | | extraction 1 | |
| | concen- | Crude after reaction | | Purity of | Yield in |
| | tration | DVM | Vanillin | DVM | solid part |
| Experiment | (%) | (%) | (%) | (%) | (%) |
| 3 | 77 | 44.8 | 49.4 | 80.6 | 38.0 |
| 5 | 70 | 45.2 | 49.7 | 82.7 | 46.2 |
| 6 | 85 | 34.6 | 44.2 | 74.1 | 30.5 |

### Experiment 7-9

Product made from phosphoric acid had dark color and yield was 46%. Alternate acid catalyst was explored. Results were shown in table 3.

TABLE 3

| | | | | | Product after | |
|---|---|---|---|---|---|---|
| | | | | | extraction 1 | |
| | | | Crude after | | Purity | Yield in |
| | | Molar | reaction | | of | solid |
| | Acid | ratio of | DVM | Vanillin | DVM | part |
| Experiment | catalyst | acid/F | (%) | (%) | (%) | (%) |
| 5 | 70% H₃PO₄ | 3.00 | 45.2 | 49.7 | 82.7 | 46.2 |
| 7 | 96% H₂SO₄ | 0.04 | 19.9 | 72.7 | 96.9 | 9.4 |
| 8 | 96% H₂SO₄ | 0.22 | 30.6 | 56.0 | 95.4 | 17.1 |
| 9 | 96% H₂SO₄ | 0.44 | 33.2 | 56.0 | 86.9 | 28.3 |

When small amount of sulfuric acid was used in experiment 7, product color was lighter but yield was 9.4%. After increasing amount of sulfuric acid, yield increased to 28% but product color turned to brown.

### Experiment 10-11

From experiment 5, molar ratio of phosphoric/formaldehyde was varied. Results were shown in table 4.

TABLE 4

| | | | | Product after | |
|---|---|---|---|---|---|
| | | | | extraction 1 | |
| | Mole | Crude after reaction | | Purity of | Yield in |
| | ratio | DVM | Vanillin | DVM | solid part |
| Experiment | H₃PO₄/F | (%) | (%) | (%) | (%) |
| 5 | 3.0 | 45.2 | 49.7 | 82.7 | 46.2 |
| 10 | 4.0 | 44.9 | 50.2 | 82.2 | 48.8 |
| 11 | 2.0 | 39.8 | 43.0 | 82.8 | 40.4 |

Less mole ratio of H₃PO_{4/}F reduced yield to 40% while mole ratio at 3-4 gave yield in range of 46-49%.

### Experiment 12

From experiment 5, solvent in extraction was changed from acetone to MIBK in experiment 12. The result was in table 5.

TABLE 5

| | | | | | Product after | |
|---|---|---|---|---|---|---|
| | | | | | extraction 1 | |
| | | | Crude after | | Purity | Yield in |
| | Mole | Type of | reaction | | of | solid |
| | ratio | solvent in | DVM | Vanillin | DVM | part |
| Experiment | H₃PO₄/F | extraction | (%) | (%) | (%) | (%) |
| 5 | 3.0 | Acetone | 45.2 | 49.7 | 82.7 | 46.2 |
| 12 | 3.0 | MIBK | 44.4 | 42.9 | 85.9 | 41.1 |

Yield from MIBK extraction was 41% while yield from acetone extraction was 46%.

### Experiment 13

From experiment 5, reaction temperature was raised from 100 to 115 deg C in experiment 13. And from exp 12, reaction time was reduced in experiment 14 and 15. Results are shown in Table 6.

TABLE 6

| Experiment | | 5 | 12 | 13 | 14 | 15 |
|---|---|---|---|---|---|---|
| Reaction temperature (° C.) | | 100 | 100 | 115 | 100 | 100 |
| Reaction time (hour) | | 8 | 8 | 8 | 6 | 4 |
| Extraction by | | Acetone | MIBK | Acetone | MIBK | MIBK |
| Product from | Purity of DVM (%) | 82.7 | 85.9 | 87.8 | 77.3 | 77.3 |
| extraction 1 | Yield in solid part (%) | 46.2 | 41.1 | 45.3 | 47.4 | 45.6 |
| Product from | Purity of DVM (%) | — | 93.6 | — | 90.1 | 90.2 |
| extraction 2 | Yield (%) | — | 41.1 | — | 47.3 | 43.8 |

### Experiment 16

Divanillylmethane (250 g, 0.79 mole), epichlorohydrin (1462 g, 15.3 mole), and benzyl triethylammonium chloride (2.5 g, 0.011 mole) were placed in a round-bottom flask. The reaction mixture was stirred and heated up to 110° C. The reaction mixture was kept at 110° C. for 4 h. Then reaction mixture was cooled down to 65° C. and kept under vacuum at 160 mbar. A solution of 50% sodium hydroxide (126.5 g, 1.58 mole) was added at a uniform rate at 65° C. under vacuum with continuous water separation. After sodium hydroxide addition was completed, the reaction was continued for 30 minutes. Then excess epichlorohydrin was removed by distillation at 110° C. under vacuum. 814.9 g of toluene and 277.3 g of water were added. The mixture was stirred at 85° C. for 15 minutes. Aqueous layer was removed. The organic layer was neutralized with 35% aqueous sodium dihydrogen phosphate solution. Toluene distillation was continued. Product was dark brown semisolid. Yield was 98.9%. The epoxy equivalent weight was 233.2 g/eq and hydrolysable chloride was 0.31%.

### Experiment 17 (Curing of DVMGE)

Divanillylmethane Glycidyl Ether (DVMGE) was characterized by determining process and performance properties using polyetheramine and a latent curing agent.

In this study, thermal and mechanical properties of DVMGE cured with two different curing agents were investigated and compared with Diglycidyl Ether Bisphenol A (DGEBA). In the first experiment, DVMGE was cured with Polyetheramine (D230). whereas DVMGE was cured with Boron Trichloride Amine Complex (Omicure BC120).

All specimens were characterized using different instruments following by their properties. Differential Scanning calorimetry (DSC) was used to analyze thermal properties, in term of glass transition temperature (Tg) according ISO 11357-2. The measurements were conducted from 30° C.-200° C. at heating rate 10° C./min. Both of 1^{st} and 2^{nd} heat scans were considered for evaluation.

Heat Distortion Temperature instrument (HDT) was used to measure the stiffness of a material under bending load and elevated temperature. This test was conducted according to ISO 75-1, 2 method A, under flexural stress 1.8 MPa.

The mechanical properties of DGEBA and DVMGE resins, was measured on the Universal Testing Machine (UTM) of INSTRON 5569 with 50 kN of max load. For flexural testing according to ISO 178, support span was set at 64 mm, and the test was performed at speed, 2 mm/min.

Furthermore, water absorption was investigated according to ISO 175. The specimens were immersed in de-ionised water under control climate condition at 23° C./50% R.H.

In the first experiment, mixing ratio error of DVMGE/D230 was investigated, in term of thermal properties, including Tg and HDT. To confirm the appropriate ratio of curing, the results are shown as Table 7.

TABLE 7
Mixing ratio error results of DVMGE/D230

| | | | Thermal properties | | | |
|---|---|---|---|---|---|---|
| Sr. | System | Mixing | Tg, ° C. | Tg, ° C. | HDT, | % |
| No. | Name | Ratio | (1^{st} Scan) | (2^{nd} Scan) | ° C. | cured |
| 1 | DVMGE/ | 100:25 | 75.63 | 84.15 | 69.90 | 101.4 |
| 2 | D230 | 100:26 | 77.59 | 86.37 | 72.50 | 105.5 |
| 3 | | 100:27 | 83.72 | 89.11 | 79.40 | 109.5 |

*curing condition: 100° C./3H + 110° C./10H

For comparative mechanical and thermal characterization, DGEBA and D230 were mixed using high speed mixer with mixing ratio 100:32 parts by weight and poured in steel molds which were pre-conditioned at curing time for hour. After that, steel molds were conditioned for curing at 100° C./3H+140° C./14H.

Whereas DVMGE which was the semi-solid state, it needed to melt at 100° C. for an hour and D230 was also conditioned at 100° C. for 10 minutes. DVMGE and D230 were mixed by hand blending at 100° C. for 2 minutes, then the mixture was poured into steel mold which was conditioned at 100° C. for an hour. were conditioned for curing at 100° C./3H+140° C./14H.

The specimens were extracted from the cured panel and characterized for flexural, thermal and water absorption tests. Comparison results between DVMGE/D230 and DGEBA/D230 are shown as Table 8.

TABLE 8
Comparative properties between
DGEBA/D230 and DVMGE/D230

| | | | Results | |
|---|---|---|---|---|
| Sr. No | Properties | Unit | DGEBA/D230 | DVMGE/D230 |

Thermal Properties

| | | | | |
|---|---|---|---|---|
| 1 | Tg (1^{st} scan) | ° C. | 93.42 | 94.41 |
| 2 | Tg (2^{nd} scan) | | 93.43 | 95.55 |
| 3 | HDT | | 80.40 | 83.50 |

Flexural Properties

| | | | | |
|---|---|---|---|---|
| 4 | Flexural Strength | MPa | 105.74 | 128.69 |
| 5 | Flexural Strain | % | 6.33 | 3.44 |
| 6 | Modulus (Elasticity) | MPa | 2890 | 4053 |

Water Absorption

| | | | | |
|---|---|---|---|---|
| 10 | 24 H. | mg. | 16.6 | 22.0 |

Regarding thermal characterization, it was found that DVMGE/D230 gave higher Tg and HDT when compared with DGEBA/D230. The flexural properties displayed that DVMGE/D230 indicated higher flexural strength and flexural modulus, whereas DGEBA/D230 presented higher strain. The water absorption, DVMGE/D230 was found to be marginally higher than DGEBA/D230.

For the second experiment, DVMGE was cured with latent curing agent, Omicure BC120 using mixing ratio 100:5 parts by weight. For specimen preparation, DVMGE was conditioned at 100° C. at for an hour whereas Omicure BC120 was conditioned at 60° C. for 10 minutes. DVMGE and Omicure BC120 were mixed by hand blending at 100° C. approximate 2 minutes, then the mixture was poured into steel molds which was conditioned at curing temperature for 1 hour. The specimens were extracted from the cured panel and characterized for flexural, thermal and water absorption tests. DVMGE/Omicure BC-120 were shown as Table 9.

TABLE 9
Properties of neat specimen of DVMGE/Omicure BC120

| | | | |
|---|---|---|---|
| Sr. No | Properties | Unit | Results |

Thermal Properties

| | | | |
|---|---|---|---|
| 1 | Tg (1^{st} scan) | ° C. | 137.82 |
| 2 | Tg (2^{nd} scan) | | 137.56 |
| 3 | HDT | | 130.30 |

Flexural Properties

| | | | |
|---|---|---|---|
| 4 | Flexural Strength | MPa | 108.2 |
| 5 | Flexural Strain | % | 2.58 |
| 6 | Modulus (Elasticity) | MPa | 4356 |

Tensile Properties

| | | | |
|---|---|---|---|
| 7 | Tensile Strength | MPa | 32.37 |
| 8 | Tensile Strain | % | 0.782 |
| 9 | Modulus | MPa | 4373 |

Water Absorption

| | | | |
|---|---|---|---|
| 10 | 24 H. | mg. | 32.9 |

Regarding the test results of DVMGE/Omicure BC120, Tg results was displayed around 137° C.

Specific embodiments: Specific embodiments are disclosed herein after:

A process for the synthesis of divanillylmethane, the process comprises reacting vanilline with an aqueous solution of formaldehyde in the presence of an acid catalyst.

Such process(s), wherein the acid catalyst is selected from a group comprising of phosphoric acid, and sulphuric acid.

Such process(s), wherein the acid catalyst is phosphoric acid having a concentration in the range of 70% to 80%.

Such process(s), wherein the aqueous solution formaldehyde has a concentration in the range of 30 to 40% wt/wt.

Such process(s), wherein the formaldehyde solution has a concentration of 35% to 40% wt/wt.

Such process(s), wherein the molar ratio of vanillin to formaldehyde is in the range of 2-6.

Such process(s), wherein the molar ratio of phosphoric acid to formaldehyde is in a range of 3-5.

Such process(s), wherein the molar ratio of sulfuric acid to formaldehyde is in a range of 0.2-0.5.

Such process(s), including preparing a mixture of vanillin and the acid catalyst, heating the mixture of vanillin and the acid catalyst to a temperature in the range of 85-100° C., adding to the heated mixture of vanillin and acid catalyst an aqueous solution of formaldehyde to obtain a reaction mixture, heating the reaction mixture to a temperature in the range of 110 to 115° C. to obtain a crude mixture of divanillylmethane, unreacted vanillin and the acid catalyst, and recovering divanillylmethane from the crude mixture.

Such process(s), wherein the reaction mixture is allowed to react for 7 to 10 hours.

Such process(s), wherein divanillylmethane is recovered from the crude mixture, in a process including removing the acid catalyst from the crude mixture to obtain an organic layer including divanillylmethane, valline and trace amounts of acid catalyst, neutralizing the trace amount of acid catalyst in the organic layer with a base, dissolving unreacted vanillin in a solvent to obtain a vanillin soluble liquid, separating the solid from the vanillin soluble liquid to obtain a first solid extract, removing traces of vanillin from the first solid extract by water extraction to obtain a second solid extract, and removing the solvent and water from the second solid extract to obtain divanillylmethane.

Such process(s), wherein the acid catalyst is recovered from the crude mixture by the process comprising allowing the crude mixture to cool to a temperature in a range of 70 to 80° C. to obtain the acid catalyst in an aqueous phase below the organic phase, and separating the acid catalyst from the organic phase.

Such process(s), wherein the trace amount of acid catalyst is neutralized by adding a sodium carbonate solution to the organic layer till the pH of the organic layer is in a range of 6 to 6.5.

Such process(s), wherein the solvent is selected from a group comprising acetone and methyl isobutyl ketone,

### INDUSTRIAL APPLICABILITY

The disclosed process allows for the production of vanillin derivative divanillylmethane (formula I) which can be used as an alternative for bisphenol A for the production of epoxy resins. The process is simple and results in obtaining higher yield. Moreover, the process uses a vanillin, which is obtained from degradation of lignin, a waste product of the paper industry.

The divanillylmethane that is obtained from the process as disclosed can be reacted with bio-based epichlorohydrin to get 100% bio-based epoxy resin. The epoxy resin had both epoxy groups and aldehydes groups. After curing with primary amine, crosslinking network from reaction of epoxy group/amine and imine bonds from reaction of aldehyde group/amine are formed. Moreover, the cured resin could be soft and reprocessed due to the presence of imine bonds.

We claim:

1. A process for the synthesis of divanillylmethane, the process comprises reacting vanillin with an aqueous solution of formaldehyde in the presence of an acid catalyst.

2. The process as claimed in claim 1, wherein the acid catalyst is selected from a group comprising of phosphoric acid, and sulphuric acid.

3. The process as claimed in claim 1, wherein the acid catalyst is phosphoric acid having a concentration in the range from 70% to 80% wt/wt.

4. The process as claimed in claim 1, wherein the aqueous solution formaldehyde has a concentration in the range of 30 to 40% wt/wt.

5. The process as claimed in claim 1, wherein the formaldehyde solution has a concentration in the range of 35% to 40% wt/wt.

6. The process as claimed in claim 1, wherein the molar ratio of vanillin to formaldehyde is in the range of 2-6.

7. The process as claimed in claim 3, wherein the molar ratio of phosphoric acid to formaldehyde is in a range of 3-5.

8. The process as claimed in claim 3, wherein the molar ratio of sulfuric acid to formaldehyde is in a range of 0.2-0.5.

9. The process as claimed in claim 1, comprising:
preparing a mixture of vanillin and the acid catalyst;
heating the mixture of vanillin and the acid catalyst to a temperature in the range of 85-100° C.;
adding to the heated mixture of vanillin and acid catalyst an aqueous solution of formaldehyde to obtain a reaction mixture;
heating the reaction mixture to a temperature in the range of 110 to 115° C. to obtain a crude mixture of divanillylmethane, unreacted vanillin and the acid catalyst; and
recovering divanillylmethane from the crude mixture.

10. The process as claimed in claim 1, wherein the reaction mixture is allowed to react for 7 to 10 hours.

11. The process as claimed in claim 1, wherein divanillylmethane is recovered from the crude mixture, in a process comprising:
separating the acid catalyst from the crude mixture to obtain an organic layer including divanillylmethane, vanilline and trace amounts of acid catalyst;
neutralizing the trace amount of acid catalyst in the organic layer with a base;
dissolving unreacted vanillin in a solvent to obtain a vanillin soluble liquid;
separating the solid from the vanillin soluble liquid to obtain a first solid extract;
removing traces of vanillin from the first solid extract by water extraction to obtain a second solid extract; and
removing the solvent and water from the second solid extract to obtain divanillylmethane.

12. The process as claimed in claim 10, wherein the acid catalyst is seperated from the crude mixture by the process comprising:
allowing the crude mixture to cool to a temperature in a range of 70 to 80° C. to obtain the acid catalyst in an aqueous phase below the organic phase; and
'separating the acid catalyst from the organic phase.

13. The process as claimed in claims 10, wherein the trace amount of acid catalyst is neutralized by adding a sodium carbonate solution to the organic layer till the pH of the organic layer is in a range of 6 to 6.5.

14. The process as claimed in claim 10, wherein the solvent is selected from a group comprising acetone and methyl isobutyl ketone.
